(19) United States
(12) Patent Application Publication
(10) Pub. No.: US 2018/0256914 A1
(43) Pub. Date: Sep. 13, 2018

(54) COMPOSITIONS AND METHODS FOR THE TREATMENT OF MUCOSITIS ASSOCIATED WITH TREATMENT-INDUCED NEUTROPENIA

(71) Applicant: KLOX TECHNOLOGIES INC., Laval (CA)

(72) Inventors: Lise HÉBERT, Montreal (CA); Michel CIMON, Montreal (CA); David OHAYON, Dollard-des-Ormeaux (CA); Nikolaos LOUPIS, Athens (GR); Remigio PIERGALLINI, San Benedetto del Tronto (IT)

(21) Appl. No.: 15/745,827

(22) PCT Filed: Jul. 21, 2016
(86) PCT No.: PCT/CA2016/050856

§ 371 Date: Jan. 18, 2018

Related U.S. Application Data
(60) Provisional application No. 62/195,268, filed on Jul. 21, 2015.

(51) Int. Cl. A61N 5/06; A61K 41/00

(52) U.S. Cl. CPC A61N 5/062; A61K 41/0057; A61N 2005/0663; A61N 2005/0609; A61N 2005/0656; A61N 2005/0606

(57) ABSTRACT

The present disclosure provides compositions and methods for treating mucositis associated with treatment-induced neutropenia in a patient. The methods comprise applying a biophotonic composition comprising a fluorescent dye and a carrier to mucosal lesions and subsequently exposing the composition to actinic light.

This application claims priority to and benefit from U.S. Provisional Patent Application 62/195,268, filed Jul. 21, 2015, the disclosure of which is incorporated herein by reference in its entirety.

### BACKGROUND OF THE DISCLOSURE

Neutropenia is a reduction in the blood neutrophil (granulocyte) count, often resulting in an increased susceptibility to various types of infections. Neutropenia can be chronic, e.g. in patients infected with HIV, or acute, e.g. in cancer patients undergoing chemotherapy, radiation therapy or immunosuppressive therapy. Neutropenia is classified by the neutrophil count and the relative risk of infection: mild (1000 to 1700 cells/μL), moderate (500 to 1000 cells/μL), or severe (<500 cells/μL). If neutropenia is severe, the risk and severity of bacterial and fungal infections increases. When neutrophil counts fall to under 500 cells/μL, endogenous microbial flora (e.g., in the mouth or gut) can cause infections. If the count falls to under 200 cells/μL, inflammatory response may be nonexistent. Although there are multiple congenital and acquired causes of neutropenia (e.g., bacterial, viral, fungal, and parasitic infections; nutritional deficiencies; copper deficiency; protein malnutrition; and immune reactions), drug therapy plays a significant role in causing neutropenia. Radiation therapy can also cause neutropenia.

Neutropenia is often concomitant with mucositis. Mucositis is the destruction of the mucosal epithelium, which may result in erythema, ulcerations and severe pain in the oral cavity and gastrointestinal tract. Mucositis often arises as a complication of cancer therapy due to the direct toxic effect to the oropharyngeal epithelium by chemotherapeutic agents, radiation therapy or a combination of the two approaches. Disruption of the oral mucosa, in severe cases ulcers, commonly leads to a debilitating pain that affects eating and often creates a need for opioid analgesics. A further complication associated with mucositis is that the lesions can act as sites of secondary infections and as portals of entry for endogenous microorganisms. In fact, in neutropenic patients, mucositis is associated with an increased risk of infection, in severe cases, culminating in systemic complications including sepsis.

Currently the treatment options for mucositis associated with treatment-induced neutropenia are very limited. Accordingly, there is still a need for new therapies effective in the treatment and prevention of mucositis associated with treatment-induced neutropenia.

### SUMMARY OF THE DISCLOSURE

The object of the present disclosure is to provide methods and uses for treating mucosal lesions associated with treatment-induced neutropenia in a patient.

In one aspect, there is provided a method of treating mucositis associated with treatment-induced neutropenia in a patient comprising: (a) applying a composition comprising a fluorescent dye and a pharmaceutically acceptable carrier on the patient's mucosal lesions; and (b) exposing the composition to actinic light having a wavelength between 400 nm and 800 nm; wherein the patient suffers from a treatment-induced neutropenia.

In certain embodiments of any of the foregoing or following, the mucositis is oral mucositis. In further embodiments, the mucositis is selected from oral mucositis, esophageal mucositis, digestive tract mucositis, gastrointestinal mucositis and combinations thereof.

In certain embodiments of any of the foregoing or following, the treatment-induced neutropenia is due to cancer treatment, anti-inflammatory treatment, anti-viral treatment, or immune-suppressive treatment. In some embodiments, the cancer treatment is radiation therapy. In some embodiments, the cancer treatment is chemotherapy. In some embodiments, the chemotherapy is myelosuppressive or myeloablative chemotherapy. In some embodiments, the chemotherapy is selected from methotrexate, 5-fluorouracil, sunitinib, sorafenib, pazpanib, afatinib, everolimus, epirubicin, doxorubicin, capecitabine and combinations thereof.

In certain embodiments of any of the foregoing or following, the patient is a cancer patient. In certain such embodiments, the patient has a hematologically-related cancer.

In certain embodiments of any of the foregoing or following, the patient suffers from a bone marrow disease.

In certain embodiments of any of the foregoing or following, the patient has received or will be receiving hematopoietic stem cell transplantation. In other embodiments, the patient has received or will be receiving bone marrow transplantation.

In certain embodiments of any of the foregoing or following, the anti-inflammatory treatment is for treatment of an auto-immune condition. In some embodiments, the auto-immune condition is selected from rheumatoid arthritis, systemic lupus erythematosus and psoriasis. In some embodiments, the anti-inflammatory treatment is an anti-TNF-α agent. In some embodiments, the anti-inflammatory treatment is selected from infliximab, adalimumab and rituximab.

In certain embodiments of any of the foregoing or following, the composition is exposed to actinic light for a period of less than about 5 minutes. In further embodiments, the composition is exposed to actinic light for a period of about 1 second to about 20 minutes. In further embodiments, the composition is exposed to actinic light for a period of less than about 5 minutes per cm² of an area to be treated. In further embodiments, the composition is exposed to actinic light for a period of about 1 second to about 5 minutes per cm² of an area to be treated. In some embodiments of any of the foregoing or following, the composition is exposed to actinic light for a period of less than 5 minutes. In further embodiments, the composition is exposed to actinic light for a period of 1 second to 20 minutes. In further embodiments, the composition is exposed to actinic light for a period of less than 5 minutes per cm² of an area to be treated. In further embodiments, the composition is exposed to actinic light for a period of 1 second to 5 minutes per cm² of an area to be treated.

In certain embodiments of any of the foregoing or following, the carrier is selected from: glucose, modified starch, methyl cellulose, carboxymethyl cellulose, propyl cellulose, hydroxypropyl cellulose, carbomer polymers, glycerin, alginic acid, sodium alginate, potassium alginate, ammonium alginate, calcium alginate, agar, carrageenan, locust bean gum, pectin, gelatin, and combinations thereof.

In certain embodiments of any of the foregoing or following, the composition further comprises an oxygen source selected from molecular oxygen, water, peroxide, and combinations thereof. In some embodiments, the peroxide is hydrogen peroxide. In some embodiments, the peroxide is carbamide peroxide. In some embodiments, the peroxide is benzoyl peroxide.

In certain embodiments of any of the foregoing or following, the fluorescent dye is Eosin Y. In other embodiments, the fluorescent dye is any one of eosin Y, rhodamine B, rhodamine WT, rhodamine G, phloxine B, rose bengal, merbromine, eosin B, fluorescein, erythrosine B, saffranin O, saffron red powder, annatto extract, brown algae extract, basic fuchsin, acid fuschin, 3,3′ dihexylocarbocyanine iodide, carminic acid, indocyanine green, crocetin, α-crocin (8,8-diapo-8,8-carotenoic acid), zeaxanthine, lycopene, α-carotene, β-carotene, bixin, fucoxanthine, methyl violet, neutral red, para red, amaranth, carmoisine, allura red AC, tartrazine, orange G, ponceau 4R, methyl red, murexide-ammonium purpurate, pyronine Y pyronine B, and combinations thereof.

In certain embodiments of any of the foregoing or following, the lesions are infected with a prokaryotic microbe. In some embodiments, the microbe is a gram-positive bacterium. In some embodiments, the microbe is a gram-negative bacterium. In some embodiments, the gram-negative bacterium is a bacillus. In some embodiments, the lesions are infected with any one of Staphylococcus aureus, Pseudomonas aeruginosa, Steptococcus spp., Enterococcus spp., Enterobacteriaceae spp., Escherichia coli, Klebsiella spp., Serratia spp., Actinomyctes spp., Porphyromona gingivalis and Stomatococcus mucilginosis.

In certain embodiments of any of the foregoing or following, the lesions are infected with a eukaryotic microbe. In some embodiments, the microbe is yeast. In some embodiments, the yeast is a Candida species. In some embodiments, the Candida species is C. albicans.

In certain embodiments of any of the foregoing or following, the patient has mild neutropenia, severe neutropenia, moderate neutropenia, acute neutropenia, or chronic neutropenia. In some embodiments, the patient has a neutrophil count of 1000 to 1700/μL. In some embodiments, the patient has a neutrophil count of 500 to 1000/μL. In some embodiments, the patient has a neutrophil count of less than 500/μL.

In certain embodiments of any of the foregoing or following, method comprises further administering a PAR-1 inhibitor, palifermin, glutamine, L-glutamine, teduglutide, sucralfate mouth rinses, iseganan, lactoferrin, mesna, trefoil factor, or vitamin D.

In certain embodiments of any of the foregoing or following, the method reduces or alleviates pain in the oral cavity, the esophageal tract, the gastrointestinal tract, the digestive tract, or combinations thereof. In some embodiments, the method concomitantly reduces or alleviates the lesion condition and an infection associated with the lesion.

In another aspect, there is provided a method of augmenting a cancer treatment regimen comprising: (a) administering chemotherapeutic agent or a radiation therapy to a cancer patient; (b) applying a composition comprising a fluorescent dye and a pharmaceutically acceptable carrier on the patient's mucosal lesions; and (c) exposing the composition to actinic light having a wavelength between 400 nm and 800 nm. In some embodiments, the chemotherapeutic agent or radiation therapy is administered before, concurrent with, or after applying the composition to the mucosal lesion.

In another aspect, there is provided a method of preventing mucositis associated with treatment-induced neutropenia in a patient comprising: (a) selecting a patient in whom a neutrophil count of less than 500 cells/μL has been determined; (b) applying a composition comprising a fluorescent dye and a pharmaceutically acceptable carrier on the patient's mucosal lesions; and (c) exposing the composition to actinic light having a wavelength between 400 nm and 800 nm.

In another aspect, there is provided the use of a composition comprising a fluorescent dye and a pharmaceutically acceptable carrier for treatment of mucositis in a patient suffering from treatment-induced neutropenia.

In another aspect, there is provided the use of a composition comprising a fluorescent dye and a pharmaceutically acceptable carrier in the preparation of a medicament for treatment of mucositis in a patient suffering from treatment-induced neutropenia.

In another aspect, there is provided the use of a composition comprising a fluorescent dye and a pharmaceutically acceptable carrier in combination with a chemotherapeutic agent or a radiation therapy for augmenting a cancer treatment regimen in a cancer patient.

In another aspect, there is provided the use of a composition comprising a fluorescent dye and a pharmaceutically acceptable carrier in the preparation of a medicament for augmenting a cancer treatment regimen wherein the cancer treatment regimen includes a chemotherapeutic agent or a radiation therapy.

In another aspect, there is provided the use of a composition comprising a fluorescent dye and a pharmaceutically acceptable carrier for preventing mucositis associated with treatment-induced neutropenia in a patient, wherein the patient has a neutrophil count of less than 500 cells/μL.

In another aspect, there is provided the use of a composition comprising a fluorescent dye and a pharmaceutically acceptable carrier in the preparation of a medicament for prevention of mucositis associated with treatment-induced neutropenia in a patient, wherein the patient has a neutrophil count of less than 500 cells/μL.

In certain embodiments of any of the foregoing or following, the composition is activatable by exposure to actinic light having a wavelength between 400 nm and 800 nm.

### BRIEF DESCRIPTION OF THE DRAWINGS

FIG. 1 shows a schematic of the testing methodology for the contact solution test of Example 1. The tested gel was approximately 2 mm thick.

FIG. 2 shows that the addition of Trolox at 2 mM neutralizes the reactive oxygen species (ROS) produced by the gel containing 12% urea peroxide (UP). UP=urea peroxide.

FIG. 3 shows effect of a biophotonic gel of the disclosure on P. aeruginosa growth in the contact solution test of Example 1.

FIG. 4 shows the results of dilution-neutralisation experiments using the contact solution test of Example 1. UP=urea peroxide.

FIG. 5 shows a schematic of the testing methodology for the no-contact test of Example 1. The tested gel was approximately 2 mm thick.

FIG. 6 shows effect of a biophotonic gel of the disclosure on P. aeruginosa growth in the no-contact test of Example 1. UP=urea peroxide.

### DETAILED DESCRIPTION OF THE DISCLOSURE

### Definitions

Before continuing to describe the present disclosure in further detail, it is to be understood that this disclosure is not limited to specific compositions or process steps, as such may vary. It must be noted that, as used in this specification and the appended claims, the singular form “a”, “an” and “the” include plural referents unless the context clearly dictates otherwise.

As used herein, the term “about” in the context of a given value or range refers to a value or range that is within 20%, preferably within 10%, and more preferably within 5% of the given value or range.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone).

“Biophotonic” means the generation, manipulation, detection and application of photons in a biologically relevant context. In other words, biophotonic compositions exert their physiological effects primarily due to the generation and manipulation of photons. “Biophotonic composition” is a composition as described herein that may be activated by light to produce photons for biologically relevant applications.

Terms “chromophore”, “fluorophore,” “fluorescent dye,” “photosensitizing agent”, “photosensitizer” and “photoactivator” are used herein interchangeably. A chromophore means a chemical compound, which when contacted by light irradiation, is capable of absorbing and re-emitting light upon excitation by light. The chromophore readily undergoes photoexcitation and then transfers its energy to other molecules.

The term “actinic light” is intended to mean light energy emitted from a specific light source (lamp, LED, or laser) and capable of being absorbed by matter (e.g., the chromophore or photoactivator defined below). In some embodiments, the actinic light is visible light.

The term “chemotherapy” refers to a category of treatment using agents/drugs that are destructive to tumor cells and certain tissues. Examples of such agents/drugs include small molecule compounds and biologics.

The term “radiation therapy” refers to a category of treatment using intense energy therapy that is destructive to tumor cells and certain tissues. Radiation therapy most often gets its power from x-rays, but it can also come from other forms of energy.

The term “oxygen source” refers to a compound capable of providing oxygen to the composition. Such compounds include molecular oxygen, water, hydrogen peroxide, carbamide peroxide, benzoyl peroxide, and combinations thereof.

The term “mucositis” refers to the painful inflammation and ulceration of the mucous membranes lining the oral cavity, the esophageal tract, the gastrointestinal tract, the digestive tract or combinations thereof usually as an adverse effect of chemotherapy and radiotherapy treatment for cancer.

The term “treatment-induced neutropenia” refers to a disorder characterized by a low level of neutrophils in peripheral blood which is caused by radiation therapy, chemotherapy or other treatment regimens which suppress bone marrow.

### Overview

Mucositis is a serious and often very painful disorder involving inflammation of the mucous membrane, with the inflammation often accompanied by infection and/or ulceration. The ulcerative lesions are painful, restrict oral intake and, importantly, act as sites of secondary infection for endogenous oral and gastrointestinal flora. Mucositis often develops as a side effect of cancer therapy, and especially as a side effect of chemotherapy and radiation therapy for the treatment of cancer. Mucositis is often concomitant with neutropenia. The neutropenia puts the patient with mucositis at significant risk for systemic infection. Patients with mucositis and neutropenia have a relative risk of septicemia that is greater than four times that of individuals without mucositis. Accordingly, there is a need for an effective therapy for mucositis associated with treatment-induced neutropenia.

Biophotonic Compositions for Treatment of Mucositis Associated with Treatment-Induced Neutropenia

The present disclosure provides compositions and methods for treating mucositis associated with treatment-induced neutropenia in a patient. The methods comprise applying a biophotonic composition comprising a fluorescent dye and a carrier to mucosal lesions and subsequently exposing the composition to actinic light. Without being bound to theory, the present compositions and methods are thought to promote healing of mucosal lesions by promoting re-epithelialization, exhibiting an antimicrobial effect, and/or reducing inflammation.

When a fluorescent dye absorbs a photon of a certain wavelength, it becomes exited. This is an unstable condition and the molecule tries to return to the ground state, giving away the excess energy. For some dyes, it is favorable to emit the excess energy as light when transforming back to the ground state. This process is called fluorescence. The peak wavelength of the emitted fluorescence is shifted towards longer wavelengths compared to the absorption wavelengths (‘Stokes’ shift′). The emitted fluorescent energy can then be transferred to the other components of the composition or to a treatment site on to which the biophotonic composition is topically applied. It is thought that the fluorescent light emitted by photoactivated dyes have therapeutic properties leading to biomodulation. Furthermore, the emitted fluorescence has a longer wavelength than the activating light and hence is able to penetrate deeper into tissue. Moreover, the generation of oxygen species generated by the photoactivation of the composition may have a physical impact on the tissue to which the composition is applied. For example, the oxygen species released in the composition following excitation may help dislodge biofilm and help in the debridement of necrotic tissue.

(a) Fluorescent Dyes

The biophotonic topical compositions of the methods and uses of the present disclosure comprise one or more fluorescent dyes. In the present disclosure, re-emission of light in the green to yellow spectrum would be advantageous, since it is a deep penetrating wavelength range, with deep absorption by the blood. This confers a strong increase on the blood flow, vasodilatation and angiokinetic phenomena. Suitable fluorescent dyes include, but are not limited to xanthene dyes, azo dyes, biological stains, and carotenoids.

Xanthene Derivatives

The xanthene group consists of 3 sub-groups that are: a) fluorenes; b) fluorones; and c) rhodoles. The fluorenes group comprises the pyronines (e.g., pyronine Y and B) and the rhodamines (e.g., rhodamine B, G and WT). Depending on the concentration used, both pyronines and rhodamines may be toxic and their interaction with light may lead to increased toxicity. Similar effects are known to occur for the rhodole dye group.

The fluorone group comprises the fluorescein dye and the fluorescein derivatives. Fluorescein derivatives include, but are not limited to, Eosin Y (tetrabromofluorescein, acid red 87, or D&C Red 22), Eosin B (acid red 91, eosin scarlet, or dibromo-dinitrofluorescein), Phloxine B (2,4,5,7-tetrabromo-4,5,6,7-tetrachlorofluorescein, D&C Red 28, or acid red 92) Erythrosine B, or simply Erythrosine (acid red 51, tetraiodofluorescein), Rose Bengal (4,5,6,7-tetrachloro-2,4,5,7-tetraiodofluorescein, or acid red 94) and Merbromine (mercurochrome).

Azo Dyes

The azo (or diazo-) dyes share the N—N group, called the azo group. Suitable azo dyes include: Methyl violet, neutral red, para red (pigment red 1), amaranth (Azorubine S), Carmoisine (azorubine, food red 3, or acid red 14), allura red AC (FD&C 40), tartrazine (FD&C Yellow 5), orange G (acid orange 10), Ponceau 4R (food red 7), methyl red (acid red 2), and murexide-ammonium purpurate.

Biological Stains

Dye molecules commonly used in staining protocols for biological materials can also be used as fluorescent dyes. Suitable biological stains include: Saffranin (Saffranin 0, basic red 2), Fuchsin (basic or acid) (rosaniline hydrochloride), 3,3′-dihexylocarbocyanine iodide (DiOC6), Carminic acid (acid red 4, natural red 4), and Indocyanin green (ICG).

Carotenoids

Carotenoid dyes can also act as photoactivators. Saffron red powder is a natural carotenoid-containing compound. Saffron contains more than 150 different compounds many of which are carotenoids (e.g., mangicrocin, reaxanthine, lycopene, and various α and β-carotenes) that show good absorption of light and beneficial biological activity. Saffron can also act as both a photon-transfer agent and a healing factor. Crocetin is another compound of saffron that was found to express an antilipidemic action and promote oxygen penetration in different tissues. The spice Annatto contains as a main constituent (70-80%) the carotenoid bixin which displays antioxidative properties. β-carotene also displays antioxidative properties. Fucoxanthine is a constituent of brown algae with a pronounced ability for photosensitization of redox reactions.

In some embodiments, the fluorescent dye is present in an amount of 0.001%-1%, 0.001%-2%, 0.001%-0.01%, 0.01%-0.1%, 0.1%-1.0%, 1%-2%, 1%-5%, 2.5%-7.5%, 5%-10%, 7.5%-12.5%, 10%-15%, 12.5%-17.5%, or 15%-20% per weight of the total composition. In some embodiments, the fluorescent dye is present in an amount of about 0.001%-1%, about 0.001%-2%, about 0.001%-0.01%, about 0.01%-0.1%, about 0.1%-1.0%, about 1%-2%, about 1%-5%, about 2.5%-7.5%, about 5%-10%, about 7.5%-12.5%, about 10%-15%, about 12.5%-17.5%, or about 15%-20% per weight of the total composition. In some embodiments, the fluorescent dye is present in an amount of 0.001%-1% per weight of the total composition. In some embodiments, the fluorescent dye is present in an amount of 0.001%-2% per weight of the total composition. In some embodiments, the fluorescent dye is present in an amount of 0.001%-0.01% per weight of the total composition. In some embodiments, the fluorescent dye is present in an amount of 0.01%-0.1% per weight of the total composition. In some embodiments, the fluorescent dye is present in an amount of 0.1%-1.0% per weight of the total composition. In some embodiments, the fluorescent dye is present in an amount of 1%-2% per weight of the total composition. In some embodiments, the fluorescent dye is present in an amount of 1%-5% per weight of the total composition. In some embodiments, the fluorescent dye is present in an amount of 2.5%-7.5% per weight of the total composition. In some embodiments, the fluorescent dye is present in an amount of 5%-10% per weight of the total composition. In some embodiments, the fluorescent dye is present in an amount of 7.5%-12.5% per weight of the total composition. In some embodiments, the fluorescent dye is present in an amount of 10%-15% per weight of the total composition. In some embodiments, the fluorescent dye is present in an amount of 12.5%-17.5% per weight of the total composition. In some embodiments, the fluorescent dye is present in an amount of 15%-20% per weight of the total composition. In some embodiments, the fluorescent dye is present in an amount of about 0.001%4% per weight of the total composition. In some embodiments, the fluorescent dye is present in an amount of about 0.001%-2% per weight of the total composition. In some embodiments, the fluorescent dye is present in an amount of about 0.001%-0.01% per weight of the total composition. In some embodiments, the fluorescent dye is present in an amount of about 0.01%-0.1% per weight of the total composition. In some embodiments, the fluorescent dye is present in an amount of about 0.1%-1.0% per weight of the total composition. In some embodiments, the fluorescent dye is present in an amount of about 1%-2% per weight of the total composition. In some embodiments, the fluorescent dye is present in an amount of about 1%-5% per weight of the total composition. In some embodiments, the fluorescent dye is present in an amount of about 2.5%-7.5% per weight of the total composition. In some embodiments, the fluorescent dye is present in an amount of about 5%-10% per weight of the total composition. In some embodiments, the fluorescent dye is present in an amount of about 7.5%-12.5% per weight of the total composition. In some embodiments, the fluorescent dye is present in an amount of about 10%-15% per weight of the total composition. In some embodiments, the fluorescent dye is present in an amount of about 12.5%-17.5% per weight of the total composition. In some embodiments, the fluorescent dye is present in an amount of about 15%-20% per weight of the total composition.

(b) Carrier

The composition for use in the methods and uses of the present disclosure comprises a pharmaceutically acceptable carrier. The carrier according to various embodiments of the present disclosure includes, but is not limited to, glucose, modified starch, methyl cellulose, carboxymethyl cellulose, propyl cellulose, hydroxypropyl cellulose, carbomer polymers, glycerin, alginic acid, sodium alginate, potassium alginate, ammonium alginate, calcium alginate, agar, carrageenan, locust bean gum, pectin, gelatin, or combinations thereof.

In some embodiments, the carrier of the methods and uses of the disclosure comprises a carbomer polymer. The carbomer polymer may be present in an amount of between about 5%-10%, 7.5%-12.5%, 10%-20%, 15%-25%, or 20%-30% of the total weight of the composition. In some embodiments, the carbomer polymer is present in an amount of between 5%-10%, 7.5%-12.5%, 10%-20%, 15%-25%, or 20%-30% of the total weight of the composition. In some embodiments, the carbomer polymer is present in the amount of about 5%-10% of the total weight of the composition, such as 5%-10% of the total weight of the composition. In some embodiments, the carbomer polymer is present in the amount of about 7.5%-12.5% of the total weight of the composition, such as 7.5%-12.5% of the total weight of the composition. In some embodiments, the carbomer polymer is present in the amount of about 10%-20% of the total weight of the composition, such as 10%-20% of the total weight of the composition. In some embodiments, the carbomer polymer is present in the amount of about 15%-25% of the total weight of the composition, such as 15%-25% of the total weight of the composition. In some embodiments, the carbomer polymer is present in the amount of about 20%-30% of the total weight of the composition, such as 20%-30% of the total weight of the composition. In some embodiments, the carrier of the methods and uses of the disclosure comprises glycerin. Glycerin may be present in an amount between about 20%-30%, 25%35%, 30%-40%, 35%-45%, 40%-50%, or 45%-55% of the total weight of the composition. In some embodiments, glycerin is present in an amount between about 20%-30%, 25%-35%, 30%-40%, 35%-45%, 40%-50%, or 45%-55% of the total weight of the composition. In some embodiments, glycerin is present in an amount between about 20%-30% of the total weight of the composition, such as 20%-30% of the total weight of the composition. In some embodiments, glycerin is present in an amount between about 25%-35% of the total weight of the composition, such as 25%-35% of the total weight of the composition. In some embodiments, glycerin is present in an amount between about 30%-40% of the total weight of the composition, such as 30%-40% of the total weight of the composition. In some embodiments, glycerin is present in an amount between about 35%-45% of the total weight of the composition, such as 35%-45% of the total weight of the composition. In some embodiments, glycerin is present in an amount between about 40%-50% of the total weight of the composition, such as 40%-50% of the total weight of the composition. In some embodiments, glycerin is present in an amount between about 45%-55% of the total weight of the composition, such as 45%-55% of the total weight of the composition.

(c) Oxygen Source

According to some embodiments, the compositions of the methods and uses of the present disclosure may contain an oxygen source. Suitable oxygen sources include water, molecular oxygen and peroxides, such as hydrogen peroxide, urea peroxide and benzoyl peroxide.

Hydrogen peroxide (H₂O₂) is a powerful oxidizing agent. The unique property of hydrogen peroxide is that it breaks down into water and oxygen and does not form any persistent, toxic residual compound. Hydrogen peroxide for use in the compositions of the methods and uses of the disclosure can be used in a gel, for example having 6% hydrogen peroxide by weight of the total composition or about 6% hydrogen peroxide by weight of the total composition. A suitable range of concentration over which hydrogen peroxide can be used in the compositions of the methods and uses of the disclosure is from about 0.1%-6%, or about 1%-12%, or about 0.5%-3%, or about less than 0.3% by weight of the total composition. In some embodiments, the compositions of the methods and uses of the disclosure comprise 0.1%-6%, or 1%-12%, or 0.5%-3%, or less than 0.3% by weight of the total composition hydrogen peroxide. In some embodiments, the compositions of the methods and uses of the disclosure comprise about 0.1%-6% by weight of the total composition hydrogen peroxide, such as 0.1%-6% by weight of the total composition hydrogen peroxide. In some embodiments, the compositions of the methods and uses of the disclosure comprise about 1%-12% by weight of the total composition hydrogen peroxide, such as 1%-12% by weight of the total composition hydrogen peroxide. In some embodiments, the compositions of the methods and uses of the disclosure comprise about 0.5%-3% by weight of the total composition hydrogen peroxide, such as 0.5%-3% by weight of the total composition hydrogen peroxide. In some embodiments, the compositions of the methods and uses of the disclosure comprise about less than 0.3% by weight of the total composition hydrogen peroxide, such as less than 0.3% by weight of the total composition hydrogen peroxide.

Urea hydrogen peroxide (also known as urea peroxide, carbamide peroxide or percarbamide) is soluble in water and contains approximately 35% hydrogen peroxide. Carbamide peroxide for use in this composition can be used as a gel, for example with 16% carbamide peroxide that represents 5.6% hydrogen peroxide. A suitable range of concentrations over which urea peroxide can be used in the compositions of the methods and uses of the disclosure is from about 0.3%-16%, or about 1%-20%, or about less than 1%, or about less than 0.5% by weight of the total composition. In some embodiments, the compositions of the methods and uses of the disclosure comprise 0.3%-16%, or 1%-20%, or less than 1%, or less than 0.5% by weight of the total composition urea peroxide. In some embodiments, the compositions of the methods and uses of the disclosure comprise about 0.3%-16% by weight of the total composition urea peroxide, such as 0.3%-16% by weight of the total composition urea peroxide. In some embodiments, the compositions of the methods and uses of the disclosure comprise about 1%-20% by weight of the total composition urea peroxide, such as 1%-20% by weight of the total composition urea peroxide. In some embodiments, the compositions of the methods and uses of the disclosure comprise about less than 1% by weight of the total composition urea peroxide, such as less than 1% by weight of the total composition urea peroxide. In some embodiments, the compositions of the methods and uses of the disclosure comprise about less than 0.5% by weight of the total composition urea peroxide, such as less than 0.5% by weight of the total composition urea peroxide. Urea peroxide breaks down to urea and hydrogen peroxide in a slow-release fashion that can be accelerated with heat or photochemical reactions. The released urea [carbamide, (NH₂)CO₂)], is highly soluble in water and is a powerful protein denaturant. It increases solubility of some proteins and enhances rehydration of the skin and/or mucosa.

Benzoyl peroxide consists of two benzoyl groups joined by a peroxide group. Benzoyl peroxide breaks down to benzoic acid and oxygen upon contact with skin, neither of which is toxic. A suitable range of concentrations over which benzoyl peroxide can be used in the compositions of the methods and uses of the disclosure is from about 2.5%-5%, or about 1%-5%, or about less than 0.3% by weight of the total composition. In some embodiments, the compositions of the methods and uses of the disclosure, benzoyl peroxide is about 2.5%-5% by weight of the total composition, such as 2.5%-5% by weight of the total composition. In some embodiments, the compositions of the methods and uses of the disclosure, benzoyl peroxide is about 1%-5% by weight of the total composition, such as 1%-5% by weight of the total composition. In some embodiments, the compositions of the methods and uses of the disclosure, benzoyl peroxide is about less than 0.3% by weight of the total composition, such as less than 0.3% by weight of the total composition.

In the methods and uses of the present disclosure, additional components may optionally be used in combination with the biophotonic compositions as described herein. Such additional components include, but are not limited to, healing factors, antimicrobials, collagens, and/or agents that promote collagen synthesis. These additional components may be applied to the lesions, prior to, at the same time of, and/or after application of the biophotonic composition. Suitable healing factors, antimicrobials, collagens, and/or agents that promote collagen synthesis are discussed below:

(d) Healing Factors

Healing factors comprise compounds that promote or enhance the healing or regenerative process of the tissues on the application site of the composition. During the photoactivation of the compositions of the methods and uses of the present disclosure, there is an increase of the absorption of molecules at the treatment site by the mucosa. An augmentation in the blood flow at the site of treatment is observed for a period of time. Suitable healing factors of the compositions, methods, and uses of the disclosure include, but are not limited to:

Hyaluronic acid (Hyaluronan or hyaluronate) is a non-sulfated glycosaminoglycan, distributed widely throughout connective, epithelial and neural tissues. It is one of the primary components of the extracellular matrix and contributes significantly to cell proliferation and migration. Hyaluronan is a major component of the epithelium, where it is involved in tissue repair. While it is abundant in extracellular matrices, it contributes to tissue hydrodynamics, movement and proliferation of cells and participates in a wide number of cell surface receptor interactions, notably those including primary receptor CD44. The hyaluronidases are a family of enzymes which degrade hyaluronan. There are at least seven types of hyaluronidase-like enzymes in humans, several of which are tumor suppressors. The degradation products of hyaluronic acid, the oligosaccharides and the very-low molecular weight hyaluronic acid, exhibit pro-angiogenic properties. In addition, recent studies show that hyaluronan fragments, but not the native high molecular mass of hyaluronan, can induce inflammatory responses in macrophages and dendritic cells in tissue injury. Hyaluronic acid is well suited to biological applications targeting the skin. Due to its high biocompatibility, it is used to stimulate tissue regeneration. Current studies show that hyaluronic acid appears in the early stages of healing to physically create room for white blood cells that mediate the immune response. It is used in the synthesis of biological scaffolds for wound healing applications and in wrinkle treatment.

Glucosamine is one of the most abundant monosaccharides in human tissues and a precursor in the biological synthesis of glycosylated proteins and lipids. The common form of glucosamine used is its sulfate salt. Glucosamine shows a number of effects including anti-inflammatory activity, stimulation of the synthesis of proteoglycans and the synthesis of proteolytic enzymes.

Allantoin is a diureide of glyoxylic acid. It has keratolytic effect, increases the water content of the extracellular matrix, enhances the desquamation of the upper layers of dead (apoptotic) skin cells, and promotes skin proliferation and wound healing.

Also, saffron can act as both a chromophore and a healing factor.

(e) Antimicrobials

Antimicrobials kill microbes or inhibit their growth or accumulation. Suitable antimicrobials for use in the methods and uses of the present disclosure include, but are not limited to, phenolic and chlorinated phenolic compounds, resorcinol and its derivatives, bisphenolic compounds, benzoic esters (parabens), halogenated carbonilides, polymeric antimicrobial agents, thazolines, trichloromethylthioimides, natural antimicrobial agents (also referred to as “natural essential oils”), metal salts, and broad-spectrum antibiotics.

(f) Collagens and Agents that Promote Collagen Synthesis

Collagen is a fibrous protein produced in dermal fibroblast cells and forming 70% of the dermis. In some embodiments of the present disclosure, the methods and uses include agents that promote collagen synthesis. Agents that promote collagen synthesis (i.e., pro-collagen synthesis agents) include, but are not limited to, amino acids, peptides, proteins, lipids, small chemical molecules, natural products and extracts from natural products.

### Methods of Use

The present disclosure provides compositions, methods, and uses for treating mucositis associated with treatment-induced neutropenia in a patient. The methods and uses comprise applying a biophotonic composition of the present disclosure comprising a fluorescent dye and a carrier to mucosal lesions and subsequently exposing the composition to actinic light. Without being bound by theory, the present compositions and methods are thought to promote healing of mucosal lesions by (i) promoting re-epithelialization, (ii) reducing inflammation and (iii) exhibiting an antimicrobial effect.

In the methods and uses of the present disclosure, any source of actinic light can be used. Any type of halogen, LED or plasma arc lamp, or laser may be suitable. The primary characteristic of suitable sources of actinic light will be that they emit light in a wavelength (or wavelengths) appropriate for activating the one or more fluorescent dyes present in the composition. In some embodiments, an argon laser is used. In some embodiments, a potassium-titanyl phosphate (KTP) laser (e.g., a GreenLight™ laser) is used. In some embodiments, a LED photocuring device is the source of the actinic light. In some embodiments, the source of the actinic light is a source of light having a wavelength between about 200 nm to about 800 nm, such as 200 nm to 800 nm. In some embodiments, the source of the actinic light is a source of visible light having a wavelength between about 400 nm to about 800 nm, such as 400 nm to 800 nm. Furthermore, the source of actinic light should have a suitable power density. Suitable power density for non-collimated light sources (LED, halogen or plasma lamps) are in the range from about 900 mW/cm² to about 2000 mW/cm², such as 900 mW/cm² to 2000 mW/cm². Suitable power density for laser light sources are in the range from about 0.5 mW/cm² to about 0.8 mW/cm², such as 0.5 mW/cm² to 0.8 mW/cm².

In some embodiments of the methods and uses of the present disclosure, the light has an energy at the subject's mucosal surface of between about 1 mW/cm² and about 500 mW/cm², or about 1 mW/cm²-300 mW/cm², or about 1 mW/cm²-200 mW/cm², wherein the energy applied depends at least on the condition being treated and the wavelength of the light. In some embodiments, the light has an energy at the subject's mucosal surface of between 1 mW/cm² and 500 mW/cm², or 1-300 mW/cm², or 1-200 mW/cm². In some embodiments, the light at the subject's mucosal surface in between about 1 mW/cm²-40 mW/cm², or about 20 mW/cm²-60 mW/cm², or about 40 mW/cm²-80 mW/cm², or about 60 mW/cm²-100 mW/cm², or about 80 mW/cm²-120 mW/cm², or about 100 mW/cm²-140 mW/cm², or about 120 mW/cm²-160 mW/cm², or about 140 mW/cm²-180 mW/cm², or about 160 mW/cm²-200 mW/cm², or about 110 mW/cm²-240 mW/cm², or about 110 mW/cm²-150 mW/cm², or about 190 mW/cm²-240 mW/cm². In some embodiments, the light at the subject's mucosal surface in between 1 mW/cm²-40 mW/cm², or 20 mW/cm²-60 mW/cm², or 40 mW/cm²-80 mW/cm², or 60 mW/cm²-100 mW/cm², or 80 mW/cm²-120 mW/cm², or 100 mW/cm²-140 mW/cm², or 120 mW/cm²-160 mW/cm², or 140 mW/cm²-180 mW/cm², or 160 mW/cm²-200 mW/cm², or 110 mW/cm²-240 mW/cm², or 110 mW/cm²-150 mW/cm², or 190 mW/cm²-240 mW/cm².

The duration of the exposure to actinic light will be dependent on the surface of the treated area, and on the severity of lesion that is being treated. The illumination of the composition may take place within seconds or even fragment of seconds, but a prolonged exposure period is beneficial to exploit the synergistic effects of the absorbed, reflected and reemitted light on the compositions of the methods and uses of the present disclosure and its interaction with the tissue being treated. In some embodiments, the time of exposure to actinic light is a period between 1 minute and 5 minutes. In some embodiments, the time of exposure to actinic light is a period between 1 minute and 4 minutes. In some other embodiments, the biophotonic composition is illuminated for a period between 1 minute and 3 minutes. In certain embodiments, light is applied for a period of 1 seconds-30 seconds, 15 seconds-45 seconds, 30 seconds-60 seconds, 0.75 minute-1.5 minutes, 1 minute-2 minutes, 1.5 minutes-2.5 minutes, 2 minutes-3 minutes, 2.5 minutes-3.5 minutes, 3 minutes-4 minutes, 3.5 minutes-4.5 minutes, 4 minutes-5 minutes, 5 minutes-10 minutes, 10 minutes-15 minutes, 15 minutes-20 minutes, or 20 minutes-30 minutes. In some embodiments of the disclosure, the composition is exposed to actinic light for a period of less than about 5 minutes, such as 5 minutes. In further embodiments, the composition is exposed to actinic light for a period of about 1 second to about 20 minutes, such as 1 second to 20 minutes. In some embodiments, the composition is exposed to actinic light for a period of less than about 5 minutes per cm² of an area to be treated, such as 5 minutes per cm² of an area to be treated. In further embodiments, the composition is exposed to actinic light for a period of about 1 second to about 5 minutes per cm² of an area to be treated, such as 1 second to 5 minutes per cm² of an area to be treated. In some embodiments, the source of actinic light is in continuous motion over the treated area for the appropriate time of exposure. In some embodiments, multiple applications of the composition and actinic light are performed. In some embodiments, composition is exposed to actinic light at least two, three, four, five or six times. In some embodiments, a fresh application of the composition is applied before exposure to actinic light.

Mucositis Associated with Treatment-Induced Neutropenia

The compositions, methods, and uses of the present disclosure may be used to treat mucositis associated with treatment-induced neutropenia. Neutropenia is a condition characterized by lower than normal levels of neutrophils in the peripheral blood. Neutropenia can be caused by intrinsic defects in myeloid cells or their precursors. Neutropenia can also result from use of certain drugs, radiation therapy, bone marrow infiltration or replacement, certain infections, or immune reactions. The most common causes include the administration of drugs, infections and marrow infiltrative processes. Numerous drugs have been shown to cause neutropenia as a side effect. Such side effects have been observed in drugs in a variety of drug classes including, for example, thyroid inhibitors, antibiotics, neuropsychotropics, cardiovascular medications, analgesics, antimalarials, nonsteroidal antiinflammatory agents, antihistamines and combinations thereof. Drugs that may induce neutropenia include small molecules such as, but are not limited to, methotrexate, 5-fluorouracil, sunitinib, sorafenib, pazopanib, afatinib, everolimus, epirubicin, doxorubicin, and capecitabine. Biologics that may induce neutropenia include, but are not limited to, infliximab, adalimumab, and rituximab.

Mean normal neutrophil counts for healthy adults are on the order of 4400 cells per microliter, with a range of 1800-7700 cells per microliter. A count of 1,000 to 500 cells per microliter is considered to be moderate neutropenia and a count of less than 500 cells per microliter is considered to be severe neutropenia. Individuals with neutrophil counts of 1,000 to 1,500 cells per microliter will normally exhibit no significant propensity for infection. These individuals, in the presence of infection, may exhibit fevers which can be managed on an outpatient basis. Individuals with moderate neutropenia may exhibit a propensity to infection. These individuals may exhibit fevers in the presence of infection which are difficult to manage. Individuals with severe neutropenia exhibit a significant propensity to infection. In some embodiments of the disclosure, the patient has a neutrophil count of 1000 to 1700/μL (mild neutropenia). In some embodiments, the patient has a neutrophil count of 500 to 1000/μL (moderate neutropenia). In some embodiments, the patient has a neutrophil count of less than 500/μL (severe neutropenia). In some embodiments, the patient has mild neutropenia, severe neutropenia, moderate neutropenia, acute neutropenia, or chronic neutropenia.

Low levels of neutrophils leave the body unprotected against bacteria and other agents that might invade the tissues. Normally, the human body is constantly exposed to large numbers of bacteria. The mucous membranes of the oral cavity, the esophageal tract, the gastrointestinal tract, and the digestive tract are inhabited with a large number of pathogens. A decrease in the number of neutrophils results in invasion of the tissues by these pathogens that are already present in the body. In such cases, painful ulcers (e.g., mucositis) may appear in the oral cavity, the esophageal tract, the gastrointestinal tract, the digestive tract or combinations thereof.

Mucositis is a serious and often very painful disorder involving inflammation of the mucous membrane, with the inflammation often accompanied by infection and/or ulceration. The ulcerative lesions are painful, restrict oral intake and, importantly, act as sites of secondary infection for endogenous oral and gastrointestinal flora. Mucositis often develops as a side effect of cancer therapy, and especially as a side effect of chemotherapy and radiation therapy for the treatment of cancer. Mucositis is often concomitant with neutropenia. The neutropenia puts the patient with mucositis at significant risk for systemic infection. Patients with mucositis and neutropenia have a relative risk of septicemia that is greater than four times that of individuals without mucositis.

The development and resolution of mucositis occurs in four interrelated phases: (i) an inflammatory/vascular response; (ii) a degenerative connective tissue and/or epithelial phase; (iii) an ulcerative/infection phase; and (iv) a healing phase.

During phase 1, the inflammatory or vascular phase, the administration of chemotherapy or radiation causes the release of the cytokines interleukin-1 (IL-1), interleukin-6 (IL-6), and tumor necrosis factor-alpha (TNF-α) from the epithelium. IL-1 induces an inflammatory response that results in increased sub-epithelial vascularity, with a consequent increase in the local levels of cytotoxic agents. Both IL-1 and TNF-α cause local tissue damage, and thereby initiate and accelerate mucositis. During phase 2, the degenerative epithelial phase, radiation and chemotherapeutic drugs affect the endothelium, the connective tissues and the dividing cells of the oral basal epithelium, resulting in reduced epithelial renewal, atrophy, and ulceration. The ulceration of the surrounding tissue is exacerbated by functional trauma and by a flood of locally produced cytokines.

Phase 3, the ulcerative/infection phase, is the most symptomatic and the most complex. This phase generally occurs at the time of the patient's maximum neutropenia. Phase 3 is characterized by the release of agents that stimulate cytokine production from bacteria on the lesions. Localized areas of full-thickness erosion develop, and a fibrous pseudomembrane sometimes grows over these areas. Secondary bacterial (gram positive or gram negative)/fungal colonization of the lesions occurs, including colonization with both gram positive and negative organisms; this stimulates cytokine release from the surrounding connective tissue, which further amplifies local tissue destruction. Pseudomonas aeruginosa, Streptococcus mitis, Steptococcus oralis, Streptococcus sanguis, vancomycin-resistant enterococci, Stomatococcus mucilginosis, Stenotrophomonas maltophilia, Candida sp., Aspergillus sp., Enterobacteriaceae spp., Escherichia coli, Klebsiella spp., Serratia spp., Actinomyctes spp., and Porphyromona gingivalis are all common infections.

During phase 4, the healing phase, epithelial proliferation and differentiation is renewed, the peripheral white blood cell count is normalized, and the local microbial flora is re-established.

The present disclosure provides a method of treating mucosal lesions associated with treatment-induced neutropenia in a patient comprising: (a) applying a composition comprising a fluorescent dye and a pharmaceutically acceptable carrier on the mucosal lesions; and (b) exposing the composition to actinic light having a wavelength between 400 nm and 800 nm. In some embodiments, the patient suffers from a medical treatment-induced neutropenia. For example, the neutropenia may be induced by cancer treatment, anti-inflammatory treatment, anti-viral treatment, or immune-suppressive treatment.

In some embodiments of the methods and uses of the disclosure, the cancer treatment is chemotherapy, radiotherapy or combinations thereof. In some embodiments of the methods and uses of the disclosure, the cancer treatment is radiotherapy. In some embodiments of the methods and uses of the disclosure, the cancer treatment is chemotherapy. In certain such embodiments, the chemotherapy may be a small molecule drug or a biologic drug, such as an antibody or polypeptide. In some embodiments, the chemotherapy is selected from methotrexate, 5-fluorouracil, sunitinib, sorafenib, pazpanib, afatinib, everolimus, epirubicin, doxorubicin, capecitabine, and combinations thereof. In some embodiments of the methods and uses of the disclosure, the chemotherapeutic agent is selected from gemcitabine (gemzar); daunorubicin; procarbazine; mitomycin; cytarabine; etoposide; venorelbine; vinca alkaloids such as vinblastine or vincristine; bleomycin; paclitaxel (taxol); docetaxel (taxotere); aldesleukin; asparaginase; busulfan; carboplatin; cladribine; camptothecin; CPT-11; 10-hydroxy-7-ethylcamptothecin (SN38); dacarbazine; S-1 capecitabine; ftorafur; 5′-deoxyfluorouridine; UFT; eniluracil; deoxycytidine; 5-azacyto sine; 5-azadeoxycytosine; allopurinol; 2-chloroadenosine; trimetrexate; aminopterin; methylene-10-deazaaminopterin (MDAM); oxaplatin; picoplatin; tetraplatin; satraplatin; platinum-DACH; ormaplatin; CI-973; JM-216 and analogs thereof; etoposide phosphate; 9-aminocamptothecin; 10,11-methylenedioxycamptothecin; karenitecin; 9-nitrocamptothecin; TAS103; vindesine; L-phenylalanine mustard; ifosphamidemefosphamide; perfosfamide; trophosphamide carmustine; semustine; epothilones A-E; tomudex; 6-mercaptopurine; 6-thioguanine; amsacrine; etoposide phosphate; karenitecin; acyclovir; valacyclovir; ganciclovir; amantadine; rimantadine; lamivudine; zidovudine; bevacizumab; trastuzumab; and combinations thereof. In some embodiments, the chemotherapy is methotrexate. In some embodiments, the chemotherapy is 5-fluorouracil. In some embodiments, the chemotherapy is sunitinib. In some embodiments, the chemotherapy is sorafenib. In some embodiments, the chemotherapy is pazpanib. In some embodiments, the chemotherapy is afatinib. In some embodiments, the chemotherapy is everolimus. In some embodiments, the chemotherapy is epirubicin. In some embodiments, the chemotherapy is doxorubicin. In some embodiments, the chemotherapy is capecitabine.

In some embodiments of the methods and uses of the disclosure, the treatment is a chemotherapy, including, but not limited to, docetaxel, mitoxantrone, and mitoxantrone hydrochloride. In some embodiments, the chemotherapy is selected from the group including, but not limited to, 20-epi-1, 25 dihydroxyvitamin D3, 4-ipomeanol, 5-ethynyluracil, 9-dihydrotaxol, abiraterone, acivicin, aclarubicin, acodazole hydrochloride, acronine, acylfiilvene, adecypenol, adozelesin, aldesleukin, all-tk antagonists, altretamine, ambamustine, ambomycin, ametantrone acetate, amidox, amifostine, aminoglutethimide, aminolevulinic acid, amrubicin, amsacrine, anagrelide, anastrozole, andrographolide, angiogenesis inhibitors, antagonist D, antagonist G, antarelix, anthramycin, anti-dorsalizdng morphogenetic protein-1, antiestrogen, antineoplaston, antisense oligonucleotides, aphidicolin glycinate, apoptosis gene modulators, apoptosis regulators, apurinic acid, ARA-CDP-DL-PTBA, arginine deaminase, asparaginase, asperlin, asulacrine, atamestane, atrimustine, axinastatin 1, axinastatin 2, axinastatin 3, azacitidine, azasetron, azatoxin, azatyrosine, azetepa, azotomycin, baccatin III derivatives, balanol, batimastat, benzochlorins, benzodepa, benzoylstaurosporine, beta lactam derivatives, beta-alethine, betaclamycin B, betulinic acid, BFGF inhibitor, bicalutamide, bisantrene, bisantrene hydrochloride, bisazuidinylspermine, bisnafide, bisnafide dimesylate, bistratene A, bizelesin, bleomycin, bleomycin sulfate, BRC/ABL antagonists, breflate, brequinar sodium, bropirimine, budotitane, busulfan, buthionine sulfoximine, cactinomycin, calcipotriol, calphostin C, calusterone, camptothecin derivatives, canarypox IL-2, capecitabine, caraceraide, carbetimer, carboplatin, carboxamide-amino-triazole, carboxyamidotriazole, carest M3, carmustine, earn 700, cartilage derived inhibitor, carubicin hydrochloride, carzelesin, casein kinase inhibitors, castanosperrnine, cecropin B, cedefingol, cetrorelix, chlorambucil, chlorins, chloroquinoxaline sulfonamide, cicaprost, cirolemycin, cisplatin, cis-porphyrin, cladribine, clomifene analogs, clotrimazole, collismycin A, collismycin B, combretastatin A4, combretastatin analog, conagenin, crambescidin 816, crisnatol, crisnatol mesylate, cryptophycin 8, cryptophycin A derivatives, curacin A, cyclopentanthraquinones, cyclophosphamide, cycloplatam, cypemycin, cytarabine, cytarabine ocfosfate, cytolytic factor, cytostatin, dacarbazine, dacliximab, dactinomycin, daunorubicin hydrochloride, decitabine, dehydrodidemnin B, deslorelin, dexifosfamide, dexormaplatin, dexrazoxane, dexverapamil, dezaguanine, dezaguanine mesylate, diaziquone, didemnin B, didox, diethyhiorspermine, dihydro-5-azacytidine, dioxamycin, diphenyl spiromustine, docetaxel, docosanol, dolasetron, doxifluridine, doxorubicin, doxorubicin hydrochloride, droloxifene, droloxifene citrate, dromostanolone propionate, dronabinol, duazomycin, duocannycin SA, ebselen, ecomustine, edatrexate, edelfosine, edrecolomab, eflomithine, eflomithine hydrochloride, elemene, elsarnitrucin, emitefur, enloplatin, enpromate, epipropidine, epirubicin, epirubicin hydrochloride, epristeride, erbulozole, erythrocyte gene therapy vector system, esorubicin hydrochloride, estramustine, estramustine analog, estramustine phosphate sodium, estrogen agonists, estrogen antagonists, etanidazole, etoposide, etoposide phosphate, etoprine, exemestane, fadrozole, fadrozole hydrochloride, fazarabine, fenretinide, filgrastim, finasteride, flavopiridol, flezelastine, floxuridine, fluasterone, fludarabine, fludarabine phosphate, fluorodaunorunicin hydrochloride, fluorouracil, fluorocitabine, forfenimex, formestane, fosquidone, fostriecin, fostriecin sodium, fotemustine, gadolinium texaphyrin, gallium nitrate, galocitabine, ganirelix, gelatinase inhibitors, gemcitabine, gemcitabine hydrochloride, glutathione inhibitors, hepsulfam, heregulin, hexamethylene bisacetamide, hydroxyurea, hypericin, ibandronic acid, idarubicin, idarubicin hydrochloride, idoxifene, idramantone, ifosfamide, ihnofosine, ilomastat, imidazoacridones, imiquimod, immunostimulant peptides, insulin-like growth factor-1 receptor inhibitor, interferon agonists, interferon alpha-2A, interferon alpha-2B, interferon alpha-N1, interferon alpha-N3, interferon beta-IA, interferon gamma-IB, interferons, interleukins, iobenguane, iododoxorubicin, iproplatm, irinotecan, irinotecan hydrochloride, iroplact, irsogladine, isobengazole, isohomohalicondrin B, itasetron, jasplakinolide, kahalalide F, lamellarin-N triacetate, lanreotide, lanreotide acetate, leinamycin, lenograstim, lentinan sulfate, leptolstatin, letrozole, leukemia inhibiting factor, leukocyte alpha interferon, leuprolide acetate, leuprolide/estrogen/progesterone, leuprorelin, levamisole, liarozole, liarozole hydrochloride, linear polyamine analog, lipophilic disaccharide peptide, lipophilic platinum compounds, lissoclinamide, lobaplatin, lombricine, lometrexol, lometrexol sodium, lomustine, lonidamine, losoxantrone, losoxantrone hydrochloride, lovastatin, loxoribine, lurtotecan, lutetium texaphyrin lisofylline, lytic peptides, maitansine, mannostatin A, marimastat, masoprocol, maspin, matrilysin inhibitors, matrix metalloproteinase inhibitors, maytansine, mechlorethamine hydrochloride, megestrol acetate, melengestrol acetate, melphalan, menogaril, merbarone, mercaptopurine, meterelin, methioninase, methotrexate, methotrexate sodium, metoclopramide, metoprine, meturedepa, microalgal protein kinase C uihibitors, MIF inhibitor, mifepristone, miltefosine, mirimostim, mismatched double stranded RNA, mitindomide, mitocarcin, mitocromin, mitogillin, mitoguazone, mitolactol, mitomalcin, mitomycin, mitomycin analogs, mitonafide, mitosper, mitotane, mitotoxin fibroblast growth factor-saporin, mitoxantrone, mitoxantrone hydrochloride, mofarotene, molgramostim, monoclonal antibody, human chorionic gonadotrophin, monophosphoryl lipid a/myobacterium cell wall SK, mopidamol, multiple drug resistance gene inhibitor, multiple tumor suppressor 1-based therapy, mustard anticancer agent, mycaperoxide B, mycobacterial cell wall extract, mycophenolic acid, myriaporone, n-acetyldinaline, nafarelin, nagrestip, naloxone/pentazocine, napavin, naphterpin, nartograstim, nedaplatin, nemorubicin, neridronic acid, neutral endopeptidase, nilutamide, nisamycin, nitric oxide modulators, nitroxide antioxidant, nitrullyn, nocodazole, nogalamycin, n-substituted benzamides, 06-benzylguanine, octreotide, okicenone, oligonucleotides, onapristone, ondansetron, oracin, oral cytokine inducer, ormaplatin, osaterone, oxaliplatin, oxaunomycin, oxisuran, paclitaxel, paclitaxel analogs, paclitaxel derivatives, palauamine, palmitoylrhizoxin, pamidronic acid, panaxytriol, panomifene, parabactin, pazelliptine, pegaspargase, peldesine, peliomycin, pentamustine, pentosan polysulfate sodium, pentostatin, pentrozole, peplomycin sulfate, perflubron, perfosfamide, perillyl alcohol, phenazinomycin, phenylacetate, phosphatase inhibitors, picibanil, pilocarpine hydrochloride, pipobroman, piposulfan, pirarubicin, piritrexim, piroxantrone hydrochloride, placetin A, placetin B, plasminogen activator inhibitor, platinum complex, platinum compounds, platinum-triamine complex, plicamycin, plomestane, porfimer sodium, porfiromycin, prednimustine, procarbazine hydrochloride, propyl bis-acridone, prostaglandin J2, prostatic carcinoma antiandrogen, proteasome inhibitors, protein A-based immune modulator, protein kinase C inhibitor, protein tyrosine phosphatase inhibitors, purine nucleoside phosphorylase inhibitors, puromycin, puromycin hydrochloride, purpurins, pyrazofurin, pyrazoloacridine, pyridoxylated hemoglobin polyoxyethylene conjugate, RAF antagonists, raltitrexed, ramosetron, RAS farnesyl protein transferase inhibitors, RAS inhibitors, RAS-GAP inhibitor, retelliptine demethylated, rhenium RE 186 etidronate, rhizoxin, riboprine, ribozymes, RH retinamide, RNAi, rogletimide, rohitukine, romurtide, roquinimex, rubiginone Bl, ruboxyl, safingol, safingol hydrochloride, saintopin, sarcnu, sarcophytol A, sargramostim, SDI1 mimetics, semustine, senescence derived inhibitor 1, sense oligonucleotides, signal transduction inhibitors, signal transduction modulators, simtrazene, single chain antigen binding protein, sizofuran, sobuzoxane, sodium borocaptate, sodium phenylacetate, solverol, somatomedin binding protein, sonermin, sparfosate sodium, sparfosic acid, sparsomycin, spiramycin D, spirogermanium hydrochloride, spiromustine, spiroplatin, splenopentin, spongistatin 1, squalamine, stem cell inhibitor, stem-cell division inhibitors, stipiamide, streptonigrin, streptozocin, stromelysin inhibitors, sulfinosine, sulofenur, superactive vasoactive intestinal peptide antagonist, suradista, suramin, swainsonine, synthetic glycosaminoglycans, talisomycin, tallimustine, tamoxifen methiodide, tauromustine, tazarotene, tecogalan sodium, tegafur, tellurapyrylium, telomerase inhibitors, teloxantrone hydrochloride, temoporfin, temozolomide, teniposide, teroxirone, testolactone, tetrachlorodecaoxide, tetrazomine, thaliblastine, thalidomide, thiamiprine, thiocoraline, thioguanine, thiotepa, thrombopoietin, thrombopoietin mimetic, thymalfasin, thymopoietin receptor agonist, thymotrinan, thyroid stimulating hormone, tiazofurin, tin ethyl etiopurpurin, tirapazamine, titanocene dichloride, topotecan hydrochloride, topsentin, toremifene, toremifene citrate, totipotent stem cell factor, translation inhibitors, trestolone acetate, tretinoin, triacetyluridine, triciribine, triciribine phosphate, trimetrexate, trimetrexate glucuronate, triptorelin, tropisetron, tubulozole hydrochloride, turosteride, tyrosine kinase inhibitors, tyrphostins, UBC inhibitors, ubenimex, uracil mustard, uredepa, urogenital sinus-derived growth inhibitory factor, urokinase receptor antagonists, vapreotide, variolin B, velaresol, versamine, verdins, verteporfin, vinblastine sulfate, vincristine sulfate, vindesine, vindesine sulfate, vinepidine sulfate, vinglycinate sulfate, vinleurosine sulfate, vinorelbine or vinorelbine tartrate, vinrosidine sulfate, vinxaltine, vinzolidine sulfate, vitaxin, vorozole, zanoterone, zeniplatin, zilascorb, zinostatin, zinostatin stimalamer, and zorubicin hydrochloride, and combinations thereof.

In some embodiments of the methods and uses of the disclosure, the patient being treated for mucosal lesions associated with treatment-induced neutropenia is a cancer patient. In some embodiments, the cancer is a hematological cancer. In some embodiments, the cancer is bone cancer. In some embodiments, the cancer is leukemia, lymphoma, rectal or colorectal cancer, breast cancer, prostate cancer, androgen-dependent prostate cancer, lung cancer, mesothelioma, head and neck cancer, esophageal cancer, gastric cancer, pancreatic cancer, gastrointestinal cancer, renal cell cancer, testicular cancer, germ cell cancer, glioma or any other primary or solid tumor. In other embodiments, the patient suffers from a bone marrow disorder. In some embodiments, the patient has received or will receive hematopoietic cell transplantation.

In some embodiments of the methods and uses of the disclosure, the treatment that induced the neutropenia is an anti-inflammatory treatment for an auto-immune condition. In some embodiments, the auto-immune condition is rheumatoid arthritis, lupus or psoriasis. In some embodiments, the anti-inflammatory treatment is an anti-TNF-α agent. In certain such embodiments, the anti-TNF-α agent is infliximab. In some embodiments, the anti-TNF-α agent is adalimumab. In some embodiments, the anti-inflammatory treatment is an anti-CD20 agent. In certain such embodiments, the anti-CD20 agent is rituximab.

The methods and uses of the present disclosure may also be used in conjunction with palliative therapies including the use of topical rinses, gels, or ointments that include lidocaine, articaine, and/or morphine, as well as other analgesic or anti-inflammatory agents. Specific examples of other agents and approaches that can be used in combination with the methods and uses of the present disclosure include, but are not limited to: PAR-1 inhibitor, palifermin, lactoferrin, mesna, trefoil factor, or vitamin D, teduglutide, sucralfate, oral cryotherapy, low-level laser therapy, chlorhexidine, amifostine, hematologic growth factors, pentoxifylline, glutamine, amifostine, antibiotic paste or pastille, hydrolytic enzymes, ice chips, benzydamine, calcium phosphate, honey, oral care protocols, povidone, zinc sulphate, flurbiprofen, diphenhydramine, granulocyte-macrophage colony-stimulating factor (GM-CSF)/granulocyte colony-stimulating factor (G-CSF), laser light therapy, and glutamine supplements.

### EXAMPLES

### Example 1

A test was conducted to determine whether or not the composition of the present disclosure has an anti-bacterial effect on a Gram-negative bacterium that may give rise to oral lesions associated with treatment-induced neutropenia. Pseudomonas aeruginosa (P. aeruginosa) was selected for this test because this Gram-negative bacterium is known to cause diseases in animals and humans. Additionally, P. aeruginosa is known to be a bacterium that may be present in an oral lesion that a patient receiving an anti-cancer treatment may be suffering from.

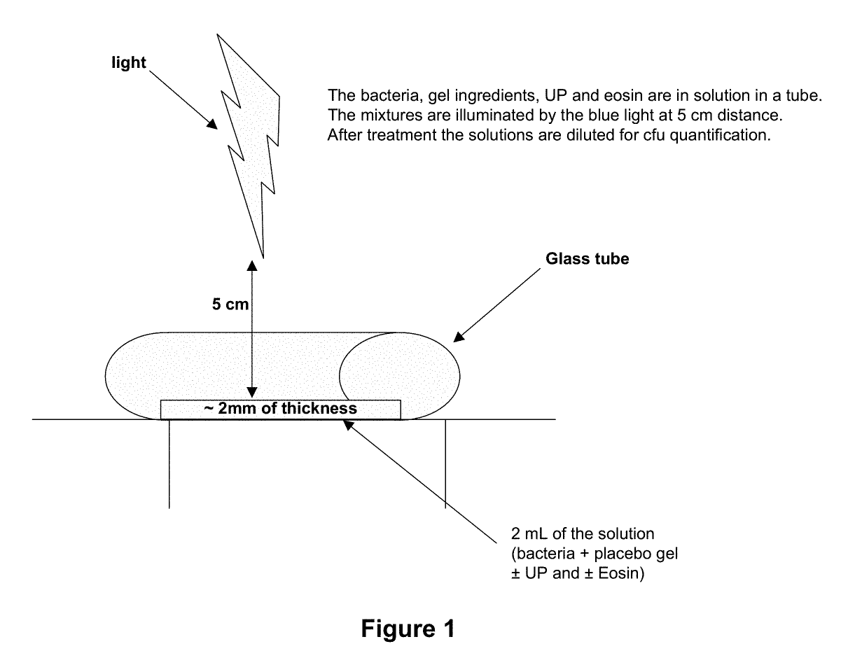

The set up for the contact solution test was done according to the schematic depicted in FIG. 1. The bacteria and the given gel (which may contain various compositions including urea peroxide and eosin) were placed in a suspension solution within a glass tube. The bacteria and given gel were illuminated with the blue light at a 5 cm distance. With respect to the amount of illumination, two different lamps with different power densities were used: a) 62 mW/cm² for the KLOX KT-D™ multi-LED lamp and b) 110 mW/cm² for the Thera™ lamp. The KT-D lamp illumination lasted 9 minutes and the Thera lamp lasted 5 minutes. The 5 minute illumination time for the Thera lamp was selected so as to provide for an illumination amount and time of exposure that was sufficient to photobleach the gel completely. Both lamps emit non-coherent light at a peak wavelength between 440 nm-460 nm.

### Log Reduction Calculation

The following formula was used to calculate the bacterial log reduction for P. aeruginosa:

Log reduction=Log₁₀(A/B) [or Log reduction=Log₁₀(A)−Log₁₀(B)], where A is the number of viable microorganisms before treatment and B is the number of microorganisms after treatment.

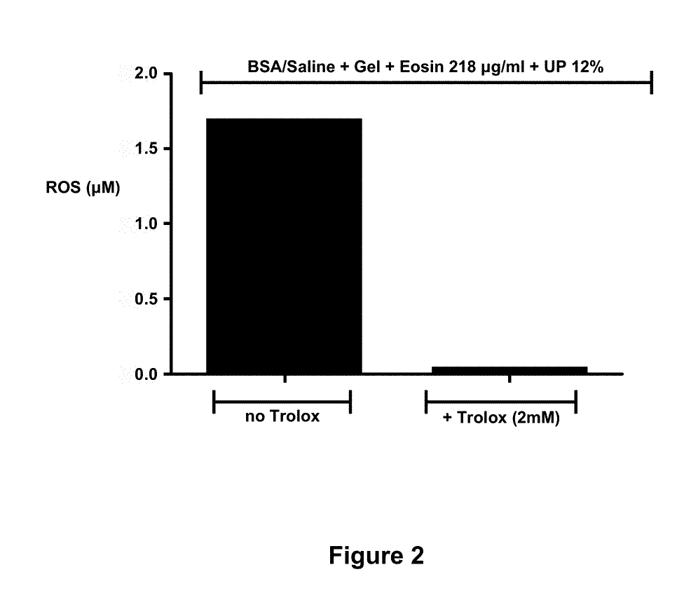

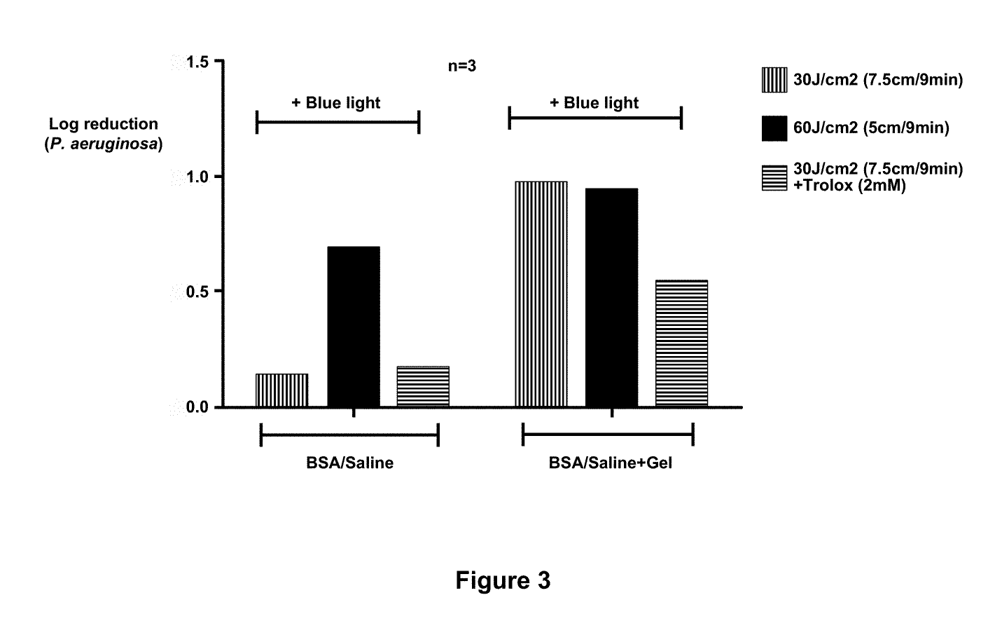

The different possibilities tested included a) illumination with light alone; b) placebo gel and illumination with light; c) gel containing eosin and illumination with light; d) gel containing eosin with varying concentrations of urea peroxide (UP) and illumination with light; and e) gel without eosin and containing varying concentrations of UP and illumination with light. In order to assess the effect that the fluorescence emanating from the illuminated gel has on the P. aeruginosa bacteria, reactive oxygen species (ROSs) were quenched via the addition of Trolox, a powerful free radical quencher; the addition of 2 mM Trolox was shown to neutralize the ROS produced by the gel containing 12% urea peroxide (see FIG. 2). Furthermore, Trolox at concentrations above 2 mM was tested and was found not to be cytotoxic to P. aeruginosa (data not shown). The log reduction of P. aeruginosa in the presence of the biophotonic gel and light was determined. In the prescence of the biophotonic gel, samples which did not contain Trolox (vertical stripes bar) were more effective at reducing the presence of P. aeruginosa as compared to the samples which contained 2 mM Trolox (horizontal stripes bar). This indicates that the peroxide in the gel may have a contributory effect (see FIG. 3).

The anti-bacterial effects of various combinations of light, gel, urea peroxide (UP), and eosin were evaluated. Experimental conditions included a) gels containing various concentrations of urea peroxide (but lacking any eosin chromophore); b) gels containing Eosin Y at 218 μg/mL (with or without 1% urea peroxide (UP)) in the presence of Trolox; and c) gels containing Eosin Y at 218 μg/mL (with or without 1% urea peroxide (UP)) in the absence of Trolox. The log reduction of P. aeruginosa under various illumination conditions is presented below in Table 1.

The results indicate that even a low concentration of peroxide in the gel can have a negative effect on the viability of the P. aeruginosa (see row 5: BSA/Saline+Gel+0.5% UP+Light). The data also indicated that the production of ROS by the illuminated peroxide and chromophore—containing gels may not have had any effect on the viability of the bacterium given the lack of difference between the non-Trolox and Trolox-containing gels (see, for example, column 2 versus column 4 of row 6: BSA/Saline+Gel+1% UP+Eosin 218 μg/mL+Light, and row 4: BSA/Saline+Gel+Eosin 218 μg/mL+Light).

TABLE 1
Log Reduction for P. aeruginosa on illumination of various gel compositions (with or
without the ROS inhibitor Trolox, and without either the chromophore, or urea peroxide (UP), or
having a particular weight percentage of UP), and comparison between the gels on being
illuminated with the blue light at either a 30 J/cm² dose or a 60 J/cm² dose.

| | | | Mean Log Reduction |
|---|---|---|---|
| | | | when appr. 30 J/cm² |
| | | Mean Log Reduction | (7.5 cm/9 min) with |
| | Mean Log Reduction | when appr. 60 J/cm² | Suppressor (2 mM |
| | when appr. 30 J/cm² | (5 cm/9 min) | Trolox) |
| Conditions | (7.5 cm/9 min) (n = 3) | (n = 3) * (n = 2) | (n = 3) * (n = 2) |
| BSA/Saline + Light | 0.14 | 0.69 | 0.16 |
| BSA/Saline + Gel + | 0.97 | 0.94 | 0.54 |
| Light | | | |
| BSA/Saline + Gel + | 1.32 | 2.12 | 1.38 |
| Eosin 218 μg/mL + | | | |
| Light | | | |
| BSA/Saline + Gel + | 3.19 | *3.40 | 2.78 |
| 0.5% UP + Light | | | |
| BSA/Saline + Gel + | 5.81 | 5.77 | 5.61 |
| 1% UP + Eosin 218 μg/mL + | | | |
| Light | | | |
| BSA/Saline + Gel + | 4.10 | 5.81 | *3.86 |
| 1% UP + Light | | | |
| BSA/Saline + Gel + | 5.71 | >6.00 | 5.28 |
| 2% UP + Light | | | |
| BSA/Saline + Gel + | >6.00 | >6.00 | 5.48 |
| 3% UP + Light | | | |
| BSA/Saline + Gel + | >6.00 | >6.00 | >6.00 |
| 6% UP + Light | | | |
| BSA/Saline + Gel + | >6.00 | >6.00 | >6.00 |
| 8% UP + Light | | | |
| BSA/Saline + Gel + | >6.00 | >6.00 | >6.00 |
| 12% UP + Light | | | |
| BSA/Saline + Gel + | >6.00 | >6.00 | >6.00 |
| 12% UP + Eosin 218 μg/mL + | | | |
| Light | | | |

Please note following conditions: BSA/Saline + Gel + 0.5% UP no Light log reduction = 0.63 and BSA/Saline + Gel + 2% UP no Light log reduction = 1.29.

Method variation: ±0.21 log

There was a low log reduction of P. aeruginosa in the control condition of gel and light. However, as the concentration of UP and eosin increased, the log reduction of P. aeruginosa increased, and the Trolox versus non-Trolox conditions were equal. Given these findings, a dilution-neutralisation experiment was conducted in order to test the gel containing Eosin Y (218 μg/mL)+12% UP wherein the potential effect of both the ROS and the peroxide were eliminated by adding Trolox (2 mM) to neutralize the ROS and adding Catalase to neutralize the peroxide. Thereafter, an assay was conducted to determine if the illuminate gel could result in a log reduction in P. aeruginosa. The controls consisted of a 3% UP gel which had Trolox and Catalase added to it, and the effect of the light illumination alone.

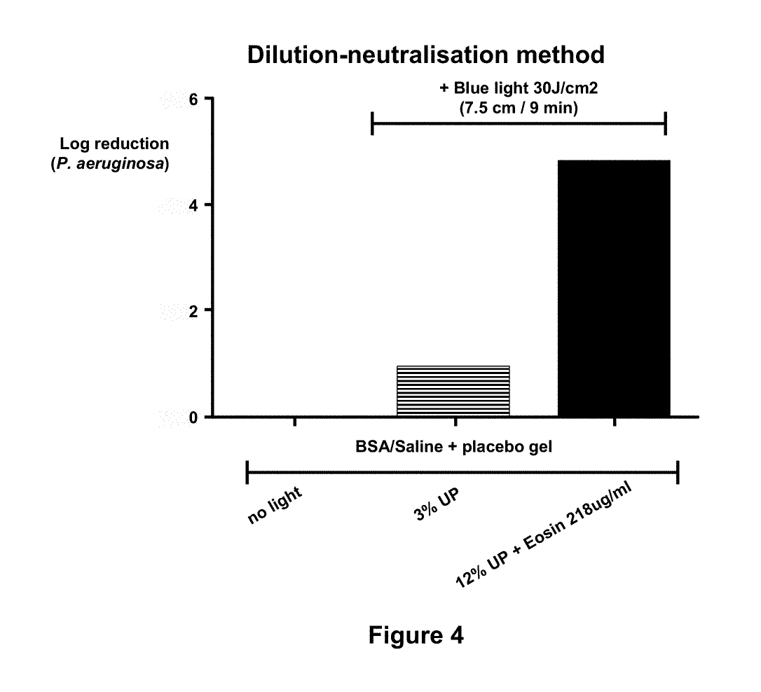

The results of the dilution-neutralisation experiment are shown in FIG. 4. The 3% UP gel+Trolox+Catalase (horizontal stripes bar) gave a reduction of less than 1 log, while the Eosin Y+12% UP gel+Trolox+Catalase (solid black bar) gave nearly a 5 log reduction. Considering that both the ROS and peroxide were neutralized, the results indicate that the fluorescence produced from the illuminated gel had a significant negative effect on the viability of the P. aeruginosa bacteria.

A further experiment was also conducted to test the anti-bacterial effect of the biophotonic composition when the gel is not in direct contact with the bacteria.

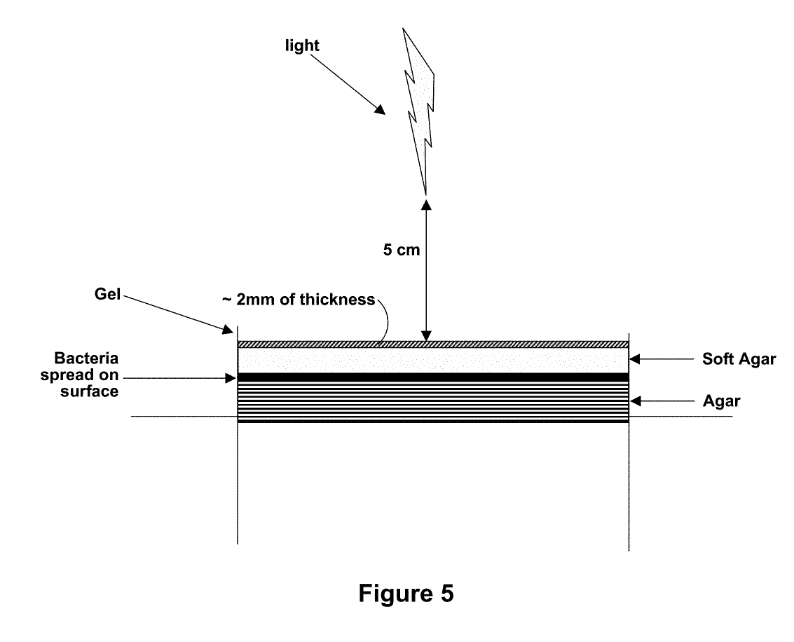

A schematic diagram of the testing methodology is presented in FIG. 5. Here, the composition is not in direct contact with the bacteria, but is separated from the bacteria by a layer of soft agar. The P. aeruginosa bacteria were plated on agar and covered by a layer of 0.5% agar (soft agar) approximately 3 mm thick. The gel to be tested was applied on the surface of the soft agar and thereafter illuminated with the blue light paced at 5 cm distance from the surface of the gel-coated soft agar. After completion of the illumination period, the gel and soft agar were removed, and the bacteria were swabbed from the agar surface for quantification and viability analysis.

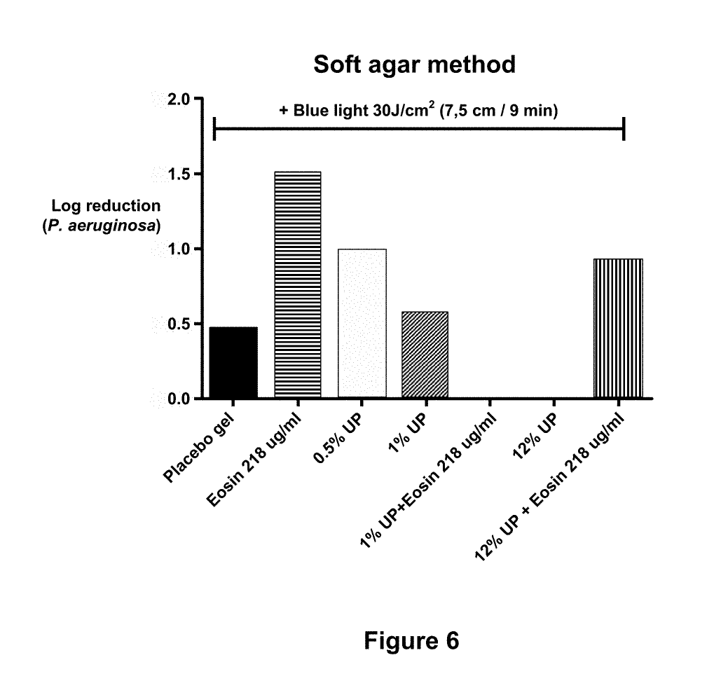

The results are presented in FIG. 6: the highest log reduction P. aeruginosa was observed in the gel containing Eosin Y only (218 μg/mL).

### EQUIVALENTS

While specific embodiments of have been discussed, the above specification is illustrative and not restrictive. Many variations will become apparent to those skilled in the art upon review of this specification and the claims below. The full scope of the disclosure should be determined by reference to the claims, along with their full scope of equivalents, and the specification, along with such variations.

### INCORPORATION BY REFERENCE

All references cited in this specification, and their references, are incorporated by reference herein in their entirety where appropriate for teachings of additional or alternative details, features, and/or technical background.

1. A method of treating mucositis in a patient suffering from treatment-induced neutropenia comprising:
(a) applying a composition comprising a fluorescent dye and a pharmaceutically acceptable carrier on the patient's mucosal lesions; and
(b) exposing the composition to actinic light having a wavelength between 400 nm and 800 nm.

2. (canceled)

3. The method of claim 1, wherein the mucositis is selected from oral mucositis, esophageal mucositis, digestive tract mucositis, gastrointestinal mucositis and combinations thereof.

4. The method of claim 1, wherein the treatment-induced neutropenia is due to cancer treatment, anti-inflammatory treatment, anti-viral treatment, or immune-suppressive treatment.

5. The method of claim 4, wherein the cancer treatment is selected from radiation therapy and chemotherapy.

6.-8. (canceled)

9. The method of claim 1, wherein the patient is a cancer patient.

10. (canceled)

11. The method of claim 1, wherein the patient suffers from a bone marrow disease.

12. The method of claim 1, wherein the patient has received or will be receiving a transplantation selected from hematopoietic stem cell transplantation and bone marrow transplant.

13. (canceled)

14. The method of claim 4, wherein the anti-inflammatory treatment is for treatment of an auto-immune condition selected from rheumatoid arthritis, systemic lupus erythematosus and psoriasis.

15. (canceled)

16. The method of claim 4, wherein the anti-inflammatory treatment is an anti-TNF-α agent.

17.-18. (canceled)

19. The method of claim 1, wherein the composition is exposed to actinic light for a period of about 1 second to about 20 minutes.

20. The method of claim 1, wherein the composition is exposed to actinic light for a period of less than about 5 minutes per cm² of an area to be treated.

21. (canceled)

22. The method of claim 1, wherein the carrier is selected from: glucose, modified starch, methyl cellulose, carboxymethyl cellulose, propyl cellulose, hydroxypropyl cellulose, carbomer polymers, glycerin, alginic acid, sodium alginate, potassium alginate, ammonium alginate, calcium alginate, agar, carrageenan, locust bean gum, pectin, gelatin, and combinations thereof.

23.-26. (canceled)

27. The method of claim 1, wherein the fluorescent dye is Eosin Y.

28. (canceled)

29. The method of claim 1, wherein the lesions are infected with one or more of a prokaryotic microbe and a eukaryotic microbe.

30.-37. (canceled)

38. The method of claim 1, wherein the patient has mild neutropenia, severe neutropenia, moderate neutropenia, acute neutropenia, or chronic neutropenia.

39. The method claim 1, wherein the patient has a neutrophil count of 1000 to 1700/μL.

40. The method claim 1, wherein the patient has a neutrophil count of 500 to 1000/μL.

41. The method claim 1, wherein the patient has a neutrophil count of less than 500/μL.

42. The method claim 1, wherein the method comprises further administering a PAR-1 inhibitor, palifermin, glutamine, L-glutamine, teduglutide, a sucralfate mouth rinse, iseganan, lactoferrin, mesna, trefoil factor, or vitamin D.

43. The method claim 1, wherein the method reduces or alleviates pain in the oral cavity, the esophageal tract, the gastrointestinal tract, the digestive tract or combinations thereof.

44.-54. (canceled)
